(19) United States
(12) Patent Application Publication
(10) Pub. No.: US 2010/0196436 A1
(43) Pub. Date: Aug. 5, 2010

(54) IMPLANTS CONTAINING DISULFIRAM AND AN ANTI-INFLAMMATORY AGENT

(76) Inventor: Lance L. Gooberman, Haddonfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/697,466

(22) Filed: Feb. 1, 2010

Related U.S. Application Data
(60) Provisional application No. 61/206,491, filed on Jan. 30, 2009.

(51) Int. Cl. A61K 31/27; A61K 31/58; A61F 2/00; A61P 25/32; A61P 25/36

(52) U.S. Cl. 424/423; 514/491; 514/171; 424/422

(57) ABSTRACT

The present invention provides an implantable composition which contains disulfiram, an anti-inflammatory agent, and optionally a pharmaceutically acceptable carrier. Methods of making the implants, and methods of using the implants to treat alcoholism and opioid dependency are also disclosed.

### CROSS-REFERENCE TO RELATED APPLICATION

This application claims the benefit of the filing date of U.S. Provisional Patent Application No. 61/206,491 filed Jan. 30, 2009, the disclosure of which is hereby incorporated herein by reference.

### BACKGROUND OF THE INVENTION

Alcohol is a commonly abused drug. According to the Diagnostic and Statistical Manual of Mental Disorders (DSM-IV), problematic alcohol use is divided into alcohol abuse and alcohol dependence.

Alcohol abuse involves recurrent alcohol consumption that negatively affects one's life, whereas alcohol dependence includes alcohol abuse and additionally symptoms of tolerance and withdrawal [McRae, et al., “Alcohol and Substance Abuse,” In: Advances in Pathophysiology and Treatment of Psychiatric Disorders: Implications for Internal Medicine, 85(d):779-801 (2001); Swift, New England J. Med. 340:1482-1490 (1999); Kick, S., Hospital Practice 34(4):95-106 (1999)]. In 1997, the estimated lifetime prevalence for alcohol abuse was 9.4% and for alcohol dependence was 14.1%, with men having significantly higher rates of dependence than women [McRae, et al., supra]. Alcohol abuse and dependence commonly lead to other problems such as alcohol-related violence, motor vehicle accidents, and medical consequences of chronic alcohol ingestion including death [McRae, et al., supra; Swift, supra].

Existing pharmacotherapies for treating alcoholism include administration of agents that inhibit the enzyme aldehyde dehydrogenase (ALDH), which is involved in the removal of acetaldehyde, a toxic metabolite of alcohol. Although multiple forms of ALDH exist. ALDH-I (also known as ALDH-2) and ALDH-II (also known as ALDH-1) are the major enzymes responsible for the oxidation of acetaldehyde. ALDH-I has a higher affinity for acetaldehyde than ALDH-II, and is thought to be the primary enzyme involved in alcohol detoxification [Keung, et al., Proc. Natl. Acad. Sci. USA 95:2198-2203 (1998)]. The discovery that 50% of the Asian population carries a mutation in ALDE-I that inactivates the enzyme, together with the low occurence of alcohol abuse in this population supports the contention that it is this isozyme of ALDH that is primarily responsible for alcohol detoxification. Recent studies also implicate ALDH-I in the metabolism of monoamine neurotransmitters such as serotonin (5-ET) and dopamine (DA) [Keung, et al., supra].

Examples of ALDH inhibitors include, e.g., disulfiram, coprine, cyanamide, 1-aminocyclopropanol (ACP), daidzin, cephalosporins, antidiabetic sulfonyl ureas, metronidazole, and any of their metabolites or analogs exhibiting ALDE-inhibiting activity including, e.g., S-methyl N,N-diethyldithiocarbamate, S-methyl N,N-diethyldithiocarbamate sulfoxide, and S-methyl N,N-diethylthiocarbamate sulfoxide. Patients who consume such inhibitors of ALDH experience mild to severe discomfort if they ingest alcohol. The efficacy of therapies using ALDH inhibitors depends on the patient's own motivation to self-administer the ALDH inhibitors, e.g., oral forms of the inhibitors, or to receive additional therapies, e.g., DEPO forms of disulfiram. In fact, patient compliance is a significant problem with these types of therapies.

Disulfiram (IUPAC: diethylcarbamothioylsulfanyl diethylaminomethanedithioate, C₁₀H²⁰N₂S₄), also known as Cronetal™, Abstenil™, Stopetyl™, Contrain™, Antadix™, Anietanol™, Exhoran™, Antabuse™, Etabuse™, Abstinyl™, Thiuranide™, Esperal™, Tetradine™, Noxal™, Tetraeti™ [Swift, supra], is a potent irreversible inhibitor of ALDH-II and inhibits ALDH-I only slightly. Recent studies suggest that the inhibition of ALDH-I by disulfiram occurs indirectly via its metabolites, e.g., S-methyl-N,N-diethylthiocarbamate sulfoxide (DETC-MeSO) [Yourick, et al., Alcohol 4:463-467 (1987); Yourick, et al., Biochem. Pharmacol. 38:413-421 (1989); Hart, et al., Alcohol 7:165-169 (1990); Madan, et al., Drug Metab. Dispos. 23:1153-1162 (1995)]. Ingestion of alcohol while taking disulfiram results in the accumulation of aldehydes, which causes tachycardia, flushing, diaphoresis, dyspnea, nausea and vomiting (also known collectively as the disulfiram or disulfiram-ethanol reaction). More simply, this drug produces sensitivity to alcohol which results in a highly unpleasant reaction when the patient under treatment ingests even small amounts of alcohol.

Although disulfiram has been available in the United States for many decades, patients frequently have difficulty complying with disulfiram treatment therapies. One reason for poor compliance is the lack of motivation for the patient to continue to take disulfiram, that is, other than self-motivation (i.e., there is no positive reinforcement for taking disulfiram). Another reason is because of the discomfort that arises if the patient ingests alcohol during disulfiram therapy [McRae, et al., supra; Swift, supra; Kick, supra]. In fact, disulfiram has not proven to be useful in maintaining long-term sobriety [Kick, supra].

### SUMMARY OF THE INVENTION

A first aspect of the present invention is directed to a subcutaneous or intramuscular implantable composition (or implant) containing a therapeutically effective amount disulfiram, an anti-inflammatory agent, and optionally, a pharmaceutically acceptable carrier. Thus, when implanted in a patient in need thereof, the disulfiram is effective as a self-sustaining delivery mechanism for its own dissolution and for delivery over a desired extended period of time. The composition may be in compressed form such as a pellet.

A second aspect of the present invention is directed to a method of making the implantable compositions (or implants), which comprises preparing an admixture of disulfiram, an anti-inflammatory agent and optionally, a pharmaceutically acceptable carrier. In embodiments wherein the composition is in the form of a pellet, the method further includes compressing the admixture, preferably substantially uniformly, into a subcutaneously or intramuscularly implantable pellet thus providing the disulfiram in concentrated form.

A third aspect of the present invention is directed to a method for the treatment of alcoholism, comprising the subcutaneous or intramuscular administration of the implant of the present invention to a patient in need thereof. Prolonged release of the disulfiram, which depending upon the amount of the drug in the implant, which in some embodiments may occur over the course of about 4 to about 12 weeks, and in some other embodiments from about 6 to about 12 weeks, effectively inhibits the enzymatic activity of aldehyde dehyrogenase, thus providing prolonged effective treatment of alcoholism.

Yet another aspect of the present invention is directed to a method for the treatment of opiate addiction, and particularly addiction to cocaine, comprising the subcutaneous or intramuscular administration the implant of the present invention to a patient in need thereof, e.g., a cocaine addict. Prolonged release of the disulfiram, which once again, depending upon the amount of the drug in the implant, may in some embodiments occur over the course of about 4 to about 12, and in some other embodiments from about 6 to about 12 weeks. Without intending to be bound by theory, it is believed that the effectiveness of the implant is based at least partially on the effect of disulfiram in inhibiting the enzyme dopamine-β-hydroxylase, which in turn increases levels of the brain chemical dopamine.

The present invention provides several advantages over existing therapies based on disulfiram. The implanation of the disulfiram eliminates the need for daily, weekly or in some cases, even monthly administrations, and ensures greater patient compliance. The steroidal anti-inflammatory compound provides for unexpectedly long-lasting dosing times, such as, for example, about 4 to about 12 weeks, and in some other embodiments, from about 6 to about 12 weeks, thus providing for particularly efficacious drug delivery periods.

### DETAILED DESCRIPTION OF TEE PREFERRED EMBODIMENTS

The amount of disulfiram contained in the composition may vary, depending upon such factors as the size of the implant and the desired time period over which release of the drug is sought. In general, amounts of disulfiram effective for the treatment of alcoholism or opiate addiction ranges from about 100 mg to about 2000 mg, and in some embodiments from about 350 mg to about 1200 mg, and in some embodiments, about 500 mg to about 800 mg. In some embodiments, the effective amount is about 380 mg, about 500 mg, about 800 mg, or about 1200 mg. For example, an implantable composition which contains polymeric microspheres and about 380 mg disulfiram may last about one month. An implantable composition in the form of a pellet containing about 500 mg disulfiram may last for about 6 to about 8 weeks. An implantable composition in the form of a pellet containing about 800 mg disulfiram may last for about 10 to about 12 weeks.

Any anti-inflammatory agent (including pharmaceutically acceptable salts and esters thereof) may be used in this invention, including without limitation, any compound that is effective to reduce blood flow to cellular elements, whether steroidal or non-steroidal, i.e., non-steroidal anti-inflammatory delivery (NSAID, e.g., salicyclate, ketorolac, naproxen, ibuprofen). By way of example only, some steroids useful herein include betamethasone dipropionate, betamethasone phosphate, betamethasone valerate, clobetasol propionate, cortisone acetate, dexamethasone phosphate acetate, dexamethasone micronized, fluocinonide, hydrocortisone acetate, hydrocortisone sulfate, methyl prednisone acetate, and triamcinolone. In some embodiments, the anti-inflammatory agent is a steroidal agent such as triamcinolone, or a pharmaceutically acceptable salt thereo including, for example, including acetonide, benetonide, furetonide, hexacetonide and diacetate.

The amount of the anti-inflammatory agent may be considered therapeutically effective in the sense that it alleviates inflammation at or around the site of administration, and may also enhance or prolong duration of the release of disulfiram. The effective amount of anti-inflammatory compound may also vary widely depending upon such diverse factors as the density of the implant, the amount of drug to be released in a desired time, and the size of the implant. In general, the amounts range from about 1.0 mg to about 1,000 mg, and in some embodiments from about 10 mg to about 80 mg.

The choice of the optional pharmaceutically acceptable carrier, e.g., which may include a binder or lubricant, is not critical. In some embodiments, the carrier is solid, e.g., a particulate solid. Examples include magnesium stearate, stearic acid, starch, and cellulose. A preferred carrier is magnesium stearate, due to its decreased solubility in physiological media providing for prolonged implant dissolution and extended drug delivery times, thus enabling the desired therapeutic disulfiram level in a patient's bloodstream for the desired amount of time.

In other embodiments, the carrier may include a water-soluble polymer or copolymer, e.g., a polyethylene glycol (PEG), a polylactic acid and copolymers thereof (polylactic acid-polyglycolic acid copolymer (PLGA)), polygalactic acid and copolymers thereof, polysaccharide (such as copolymers of lactose and galactose), and preferably poly-lactide or poly-lactide-co-glycolide polymers (PLG)(e.g., a 75:25 polylactide-co-glycolide), which entrap or encapsulate the disulfiram. In these embodiments, the implantable compositions can be made using techniques such as spray drying polymer-drug mixtures, emulsion-based technologies, coacervation-based technologies, film-casting and extrusion-based technologies. Examples of more specific techniques that may be useful include the MEDISORB® technology, as described in U.S. Pat. Nos. 6,264,987, 5,654,008 and 5,792,477; the PROLEASE® technology, as described in U.S. Pat. No. 6,358,443; the technologies described by Southern Research Institute, as described in U.S. Pat. Nos. 6,306,425 and 5,407,609; and the technologies described by Alza Corp., including the ALZAMER® Depot injection technology. Alternatively, instead of incorporating disulfiram and the anti-inflammatory agent into polymeric particles, the drugs may be formulated into an implantable composition by entrapping them microparticles prepared, for example, by coacervation techniques or by interfacial polymerization (for example, hydroxymethylcellulose or gelatine-microcapsules and poly-(methylmethacrylate) microcapsules, respectively), in colloidal drug delivery systems (for example, liposomes, albumin, microparticles, microemulsions, nanoparticles, and nanocapsules), or in macroemulsions. See also, U.S. Patent Application Publication 20050245558.

In these emobodiments wherein the carrier includes a water-soluble polymer or co-polymer, the carrier may also include a vehicle suitable for subcutaneous or intramuscular injection. Representative vehicles include biocompatible and pharmaceutically acceptable (e.g., aqueous buffered) solutions. In these embodiments, suitable excipients that may or may not be present may, for example, increase or decrease the rate of release of the agent. Ingredients which can substantially increase the rate of release include pore forming agents and other agents that facilitate polymer degradation. For example, the rate of polymer hydrolysis is increased in non-neutral pH. Therefore, an acidic or a basic excipient such as an inorganic acid or inorganic base can be added to the polymer solution used to form the microparticles, to alter the polymer erosion rate.

In these embodiments, especially wherein the implant also contains a steroidal anti-inflammatory agent, the dissolution of the polymer in physiological fluid is further delayed by the hydrophobicity of the steroid, thus further increasing the duration of the implant.

The amount of carrier present in the implant generally ranges from about 10% to about 90% by weight, based on the total weight of the implant. Typically, lesser amounts of non-polymeric carriers such as magnesium stearate are used compared to polymeric carriers.

The overall weight of the implants, particularly in the embodiments wherein they are in the form of a compressed pellet, generally ranges from about 500 mg to about 1500 mg.

In contrast to the embodiments wherein the disulfiram is formulated in polymeric microspheres, the inventive implants that are in the form of pellets may be made using any conventional device, such as a three-ton Arbor press. Typically, in order to make a pellet of a given weight, slightly more (e.g., about 10-15%) of the mixture of the disulfiram, steroidal anti-inflammatory agent and optional carrier should be used. Using a tarred scale, that initial amount is measured (to allow for chipping of the pellet during the manufacturing process and drift of the scale) and is then placed into a custom-shaped die mold, typically in the order of about 4 and ½ inches in height (see, e.g., FIG. 1 in U.S. Pat. No. 6,203,813). Relative dimensions (millimeters) of a die mold customized for use in accordance with the invention will vary, depending upon the desired shape of the pellet. The die mold is part of a tooling apparatus that also contains a punch and a base, all preferably made of hardened 440 steel.

Although the shape of the implants is not critical, in general they are cylindrical in nature. Diameter of the implants generally ranges from about 0.75 cm to about 1.5 cm, and in some embodiments from about 0.8 cm to about 1.5 cm. The dimensions preferably vary by about 10% without having much impact on the quality of the pellet produced. Using the above-described tooling apparatus, pellets having a length ranging from about 1.75 mm to about 1.85 mm may be produced, assuming proper amount of compression, which is preferably applied in a substantially uniform manner, is used on the press, e.g., at least about 2,000 psi.

There are many factors that affect the quality of the pellets. The most important of these is the inner surface of the die mold. It is preferably smooth and polished; otherwise an inordinate number of pellets will break during extraction. During the manufacture, if this becomes an acute problem, it is an indication the die needs to be resurfaced and polished. Die molds made of hardened 440-hardened stainless steel require resurfacing less often, and tend to produce a superior pellet. The compressing piston should also be made of hardened surgical steel and fit tightly against the walls of the die to prevent caking of the disulfiram around it and against the cylinder walls (which typically has a maximum clearance of about 0.0005 inches). Small granules also make a harder pellet less prone to break than the powder form of the preparation, or from large chunks obtained after the first compression. Thus using a screen allowing only particles that are 1 mm or less is employed. The entire volume of the weight of the pellet should fit into the compression cylinder of the die with minimal hand tamping, and without the need to partially compress the contents using the machine.

Finally, extraction of the pellet should be accomplished in the same direction as compression. Failure to follow these guidelines may result in a pellet that is at greater risk for breaking into fragments during extraction, placement into the insertion device, packaging and shipping, during insertion into the patient, or during the patient's usual daily activity once home. There is evidence to suggest tissue absorption of the pellet occurs more quickly if the pellet becomes fragmented after it is inserted, thus decreasing the therapeutic longevity of the pellet.

The implants produced in accordance with this invention should be sterilized before insertion into a body portion. In one embodiment, each pellet is placed into a self-sealing pouch (e.g., commercially available from Moore Medical) and labeled with the proper identification and lot number. Once a sufficient amount of packets have been prepared, they are subjected to gamma irradiation sterilization (e.g., about 7 kilorads for about 6 hours). They are stamped indicating they have been sterilized with gamma irradiation. The pellet is then ready for clinical use.

The implantable compositions of the present invention may be administered to a patient in need thereof, e.g., an alcoholic or an opioid addict, e.g., a cocaine addict, in accordance with standard medical procedures. In the case of pellets, for example, an incision of about 1-2 cm is made, usually in the area of the triceps (but other cites can be just as easily used), followed by implantation of the implant into the subcutaneous or muscular tissue. The wound is then closed. Implantable compositions in the form of polymeric microspheres may be injected subcutaneously or intramuscularly as well, e.g., in the gluteal area. The implantable compositions of the present invention may be administered once, or more than once, e.g., monthly or every 4-12 weeks. The treatment may be continued indefinitely or terminated when clinically appropriate, as determined for example by a medical professional.

The invention will now be described in accordance with the following non-limiting working examples.

### EXAMPLE 1

### Pellet Manufacture

Pellet:

### Disulfiram: 500 mg

Magnesium steate: 50 mg
Triamcinolone acetonide: 5 mg.

The amount of disulfiram was sifted through a 250 μm filtering screen to break up large aggregates and provide a medium for the uniform distribution of the other components of the mixture. The amount of triamcinolone acetonide was also sifted through a 250 μm filtering screen, and then added to the disulfiram. The amount of magnesium stearate was also sifted through a 250 μm filtering screen, and then added to the disulfiram and triamcinolone acetonide mixture, and mixed well The resultant, final mixture was then placed in a three-ton Arbor press, and compressed to form the pellet.

### EXAMPLE 2

### Manufacture of the Insertion Device

An insertion device useful and preferred for inserting the inventive drug delivery devices, for example, the illustrated pellets, is described below.

Materials:
3 cc syringes (Becton Dickinson PrecisionGlide®)
Heavy duty blade cutters
Alcohol flame

Procedure:

Pull back the plunger on the syringe to the 1.5 cc mark, then carefully heat the area of the syringe between the 1 cc mark and the hub. The barrel should be hot to the touch, but not beginning to melt. A faint discoloration of the plastic may appear.

Position the syringe such that the finger rest opposite the 1 cc mark is pointed toward toward the user while grasping the syringe by the needle covering.

Use the heavy cutters to cut the tip of the syringe off on an angle using the 1 cc line and the tip of the hub as landmarks. Discard the end and needle into the sharp container.

If cut has been made properly, the physician using the device will be allowed to use the graduations from the syringe as a measure as to how far under the skin the device is.

### Placement of the Pellets into the Insertion Device

Materials:
Prepared insertion device
Alcohol flame
Dilator/impactor
Prepared pellets

Procedure:

Heat the insertion device over the alcohol flame until hot and pliable, but not melting.

Dilate the tip of the device with the dilator by inserting it approximately 5 mm into the barrel. Let the device cool for 15 seconds before removing the dilator.

Carefully place the pellet narrow end first inside the barrel of the insertion device. Use the dilator to move it completely into the barrel, passed the proximal lip of the device.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An implant comprising a therapeutically effective amount of disulfiram, an anti-inflammatory agent, and optionally, a pharmaceutically acceptable carrier.

2. The implant of claim 1, wherein disulfiram is present in said implant in an amount of about 350 mg to about 1200 mg.

3. The implant of claim 2, wherein disulfiram is present in an amount of about 500 mg.

4. The implant of claim 3, wherein disulfiram is present in an amount of about 800 mg.

5. The implant of claim 3, wherein disulfiram is present in an amount of about 1200 mg.

6. The implant of claim 1, wherein said anti-inflammatory agent comprises triamcinolone, or a pharmaceutically acceptable salt thereof.

7. The implant of claim 1, wherein said anti-inflammatory agent is present in an amount of about 10 mg to about 80 mg.

8. The implant of claim 1, further comprising a pharmaceutically acceptable carrier.

9. The implant of claim 8, wherein said carrier comprises magnesium stearate.

10. The implant of claim 8, wherein said carrier comprises a water-soluble polymer or copolymer.

11. The implant of claim 10, wherein said water-soluble polymer or copolymer comprises a polyethylene glycol, polylactic acid or a copolymer thereof, a polygalactic acid or copolymer thereof, or a polysaccharide.

12. The implant of claim 10, wherein said water-soluble polymer comprises polylactic acid-polyglycolic acid copolymer (PLGA).

13. The implant of claim 10, wherein said water-soluble polymer comprises a poly-lactide-co-glycolide copolymer.

14. The implant of claim 1, which has a diameter of about 0.75 cm to about 1.5 cm.

15. The implant of claim 1, which has a diameter of about 0.80 cm to about 1.5 cm.

16. A process of manufacturing a disulfiram implant of claim 1, comprising the steps of:
(a) providing a therapeutically effective amount of disulfiram;
(b) providing an effective amount of an anti-inflammatory agent; and optionally
(c) providing a pharmaceutically acceptable carrier; and
(d) admixing said disulfiram and said anti-inflammatory agent and optionally said pharmaceutically acceptable carrier.

17. The method of claim 16, wherein the implant is a pellet and the method further comprises (e) applying substantially uniform pressure to the admixture of step (d) in order to obtain a disulfiram implant in the form of a pellet.

18. A method for treating alcoholism, comprising:
subcutaneously or intramuscularly administering the implant of claim 1 to a patient in need thereof.

19. A method for treating opioid addiction, comprising subcutaneously or intramuscularly administering the implant of claim 1 to a patient in need thereof.

20. The method of claim 19, wherein said patient is a cocaine addict.

21. The method of claim 19, wherein said administering is subsequent to a step of detoxifying said patient.
